SEQUENCE LISTING (1 sequence)

SEQ ID NO: 1 - protein, 17 aa, Artificial sequence
  Synthetic peptide
PGPPSPAKRPKLSSPAC

(19) United States
(12) Patent Application Publication
(10) Pub. No.: US 2021/0308133 A1
(43) Pub. Date: Oct. 7, 2021

(54) METHODS AND COMPOSITIONS FOR INHIBITING MUSCLE WASTING

(71) Applicant: THE BOARD OF REGENTS OF THE UNIVERSITY OF TEXAS SYSTEM, Austin, TX (US)

(72) Inventors: Yi-Ping LI, Houston, TX (US); Guohua ZHANG, Houston, TX (US)

(21) Appl. No.: 17/223,507

(22) Filed: Apr. 6, 2021

Related U.S. Application Data
(60) Provisional application No. 63/005,776, filed on Apr. 6, 2020.

(51) Int. Cl. A61K 31/506; A61K 45/06; A61P 35/00; A61P 21/06

(52) U.S. Cl. CPC A61K 31/506; A61P 21/06; A61P 35/00; A61K 45/06

(57) ABSTRACT

Method of reducing or preventing muscle loss, such as from cancer-induced cachexia, p38β MAPK inhibitors are provided. Compositions for use in such methods are also provided.

This application claims the benefit of U.S. Provisional Patent Application No. 63/005,776, filed Apr. 6, 2020, the entirety of which is incorporated herein by reference.

### FEDERAL FUNDING STATEMENT

The invention was made with government support under Grant No. AR067319 awarded by the National Institutes of Health. The government has certain rights in the invention.

### INCORPORATION OF SEQUENCE LISTING

The sequence listing that is contained in the file named “UTSHP0365US_ST25.txt”, which is 1 KB (as measured in Microsoft Windows®) and was created on Apr. 5, 2021, is filed herewith by electronic submission and is incorporated by reference herein.

### BACKGROUND OF THE INVENTION

### 1. Field of the Invention

The present invention relates generally to the fields of molecular biology, pharmaceutical chemistry, oncology and medicine. More particularly, it concerns preventing or reducing muscle loss in cancer and other catabolic diseases.

### 2. Description of Related Art

Cachexia is a metabolic disorder featuring a progressive loss of muscle mass resulting in muscle weakening and loss of function (muscle wasting). Muscle wasting is often associated with medical conditions, such as but not limited to: cancer; sepsis; AIDS; chronic heart failure; chronic kidney disease; Chronic Obstructive Pulmonary Disease; neuromuscular diseases (such as, but, not limited to Amyotrophic Lateral Sclerosis; Muscular Dystrophy; Multiple Sclerosis; spinal muscular atrophy) and muscle loss associated with aging (sarcopenia).

Weight loss is the hallmark of many progressive acute or chronic disease state. In its extreme form, it involves a significant lean body mass (including skeletal muscle) and fat loss. Skeletal muscle provides a fundamental basis for human function, enabling locomotion and respiration, as well as heat generation, bone maintenance, metabolism and immunity (production of antibodies requires amino acids). Muscle wasting is related to a poor quality of life and increased morbidity/mortality. Two common conditions characterized by a loss of skeletal muscle mass are sarcopenia and cachexia, which inflict a huge percentage of the human populations.

Cancer has been increasingly recognized as a systemic disease that causes disorders in multiple organs that are not resided by cancer per se. Cachexia is a metabolic syndrome seen approximately in 60% of cancer patients ¹. Cachexia is defined as a multifactorial syndrome characterized by an ongoing loss of skeletal muscle mass (with or without loss of fat mass) that cannot be fully reversed by conventional nutritional support and leads to progressive functional impairment. The clinical features of cachexia include weight loss, inflammation, insulin resistance, and increased muscle protein breakdown². Not only does cachexia increases patients' morbidity and mortality through systemic wasting, but it also decreases the efficacy while increasing the toxicity of chemotherapy ³. Consequently, cachexia is the direct cause of approximately ⅓ of cancer-related deaths ⁴. Thus, cachexia is a major determinant for the survival of cancer patients. Cachexia must be managed, because preserving muscle and body mass could promote response to cancer treatment, improve patient physical condition to withstand cancer treatment, and prolong survival. However, there has been no established treatment for cancer cachexia. In 2019, 1,762,450 new cancer cases and 606,880 cancer deaths were projected in the United States ⁵. The long standing and unmet medical need for treating cancer cachexia is substantial. Therefore, one of the Provocative Questions issued by National Institute of Health this year (RFA-CA-20-004) PQ6 is “How can cancer cachexia be reversed”. Described herein are methods and compositions for treating muscle wasting in, among other conditions, cancer-associated cachexia.

### SUMMARY OF THE INVENTION

Thus, in accordance with the disclosure, there is provided a method of reducing or preventing muscle loss in subject comprising administering a composition comprising an effective amount of a p38β MAPK inhibitor to the subject. The subject may have a muscle wasting disease, such as a cancer associated with muscle wasting. The muscle wasting may be skeletal muscle wasting and/or cardiac muscle wasting. The subject may have or have been diagnosed with, cachexia, such as cancer-induced cachexia and/or acute cachexia. The subject may have or may have been diagnosed with, cachexia induced by kidney failure, heart failure, COPD or arthritis. The subject may have or may have been diagnosed with, muscular dystrophy, such as Duchenne muscular dystrophy, polymyositis, dermatomyositis, Guillain-Barre syndrome, amyotrophic lateral sclerosis, multiple sclerosis or spinal muscular atrophy. The subject may be a human subject or a non-human animal subject.

The p38β MAPK inhibitor may be a selective p38β MAPK inhibitor, such as a p38β MAPK inhibitor having at least 2-, 3-, 4-, 5-, 10-, 20- or 50-times more active on p38β MAPK as compared to p38α MAPK. Alternatively, the p38β MAPK inhibitor may be a pan-p38 MAPK inhibitor. The p38β MAPK inhibitor may comprise Nilotinib. The p38β MAPK inhibitor may be administered more than once, such as daily, every other day, twice a week, weekly, every other week, or monthly. The method may further comprise treating said subject with a second anti-muscle wasting therapy.

In another embodiment, there is provided a method of treating a subject comprising: (a) identifying a subject having cancer-induced cachexia; and (b) administering a composition comprising an effective amount of a p38β MAPK inhibitor to the subject. The cancer may be lung cancer, breast cancer, ovarian cancer, cervical cancer, testicular cancer, colon cancer, stomach cancer, heady & neck cancer, pancreatic cancer, liver cancer, skin cancer, brain cancer, bladder cancer, bone cancer. The subject may have or have been diagnosed with cachexia. The subject may be a human subject or a non-human animal subject.

The p38β MAPK inhibitor may be a selective p38β MAPK inhibitor, such as a p38β MAPK inhibitor having at least 3-, 10-, 20- or 50-times more active on p38β MAPK as compared to p38α MAPK. Alternatively, the p38β MAPK inhibitor may be a pan-p38 MAPK inhibitor. The p38β MAPK inhibitor may comprise Nilotinib. The p38β MAPK inhibitor may be administered more than once, such as daily, every other day, twice a week, weekly, every other week, or monthly. The method may further comprise treating said subject with a second anti-cancer therapy, such as one or more of radiation therapy, chemotherapy, immune therapy, toxin therapy, hormonal therapy and/or surgery.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-C: LLC-induced muscle wasting requires site-specific phosphorylation of 300 on Ser-12 to activate C/EBPβ. (FIG. 1A) LLC induces p300 phosphorylation on Ser-12 and this reaction is critical to p300-mediated acetylation and activation of C/EBPβ. C2C12 myoblasts were transfected with a plasmid encoding phosphorylation-defective mutant of p300, p300-S12A or p300-S89A, or empty vector as control. After differentiation, myotubes were treated with LLC cell conditioned medium (LCM) or conditioned medium of non-tumorigenic NL20 cells for 2 h. Cell lysates were analyzed by Western blotting. (FIG. 1B) Overexpression of p300-S12A attenuates muscle weight loss in LLC tumor-bearing mice. Seven days after LLC implantation, the tibialis muscle (TA) was transfected with the p300-S12A-encoding plasmid. The contralateral TA was transfected with empty vector as control. After the development of cachexia, TA muscles were collected and weighed on day 21. Overexpression of transfected plasmid was confirmed by Western blotting analysis against p300. (FIG. 1C) Overexpression of p300-S12A blunts the loss of myofiber mass in LLC tumor-bearing mice. H&E-stained TA cross sections were analyzed for myofiber cross-sectional area. * indicates a statistically significant difference (p<0.05) determined by one-way ANOVA (FIG. 1A), paired Student t-test (FIG. 1B) or Chi-square test (FIG. 1C).

FIG. 2A-B: TLR4 mediates LLC-induced p300 phosphorylation on Ser-12. (FIG. 2A) TLR4 is critical to p300 phosphorylation on Ser-12 and C/EBPβ acetylation on Lys-39 in LCM-treated myotubes. C2C12 myoblasts were transfected with siRNA specific for TLR4 or scrambled control siRNA. Differentiated myotubes were treated with LCM for 2 h. Cell lysates were analyzed by Western blotting. (FIG. 2B) TLR4 is required for p300 phosphorylation on Ser-12 and C/EBPβ acetylation on Lys-39 in the muscle of LLC tumor-bearing mice. Wild-type (WT) and TLR4−/− mice (n=6) were implanted with LLC cells or injected with PBS as control. In 21 days, TA muscle were analyzed by Western blotting. * indicates a statistically significant difference (p<0.05) determined by oneway ANOVA.

FIG. 3A-E: Diverse types of cancer induce p300 phosphorylation on Ser-12 through p38β MAPK. (FIG. 3A) LCM-induced Ser-12 phosphorylation of p300 in myotubes requires p38β MAPK. C2C12 myoblasts were transfected with siRNA specific for p38a MAPK or p38β MAPK, or scrambled control. Differentiated myotubes were treated with LCM for 2 h, the cell lysates were analyzed by western blotting as indicated. (FIG. 3B) KCM induces Ser-12 phosphorylation of p300 in myotubes in a p38β MAPK-dependent manner. C2C12 myotubes transfected with p38β MAPK specific or scrambled siRNA were treated with KCM for 2 h, the cell lysates were analyzed by western blotting as indicated. (FIG. 3C) Overexpression of constitutively active p38β MAPK is sufficient to phosphorylate Ser-12 of p300. C2C12 myoblasts were transfected with plasmid encoding a constitutively active mutant of p38α MAPK or p38p MAPK. Differentiated myotubes were analyzed by Western blotting as indicated. (FIG. 3D) LCM stimulates an interaction between p300 and p38β MAPK, but not p38α MAPK. C2C12 myotubes were transfected with either p38α or p38β MAPK-specific siRNAs, and then treated with LCM for 2 h. Immunoprecipitation of p38 MAPK from the cell lysates was performed with pre-immune IgG as a control. Precipitates were then analyzed by Western blotting to verify the knockdown effects and coprecipitation of p300. (FIG. 3E) Activation of p300 in the muscle of LLC tumor-bearing mice requires p38β MAPK. LLC cells were implanted to p38β MAPK muscle-specific knockout mice (p38β mKO) and p38β MAPK floxed mice (p38βf/f). In 21 days, mice were euthanized and TA lysates were analyzed by Western blotting as indicated. * signifies a statistically significant difference (p<0.05) determined by one-way ANOVA.

FIG. 4A-D: Selective inhibition of p38β MAPK by nilotinib abrogates LLC-induced myotube catabolism without inhibiting myogenesis. (FIG. 4A) Nilotinib is ˜20-fold more potent than SB202190 in the inhibition of LLC-induced activation of p300. C2C12 myotubes were pre-treated with either nilotinib or SB202190 (SB) at indicated doses for 30 mins followed by 2 h of LCM treatment. Activation of p300 and C/EBPβ were analyzed by Western blotting as indicated. (FIG. 4B) Nilotinib abrogates LCC-induced loss of MHC in myotubes. Myotubes pre-treated with 500 nM of nilotinib or DMSO were incubated with LCM for 72 hrs. Cell lysates were analyzed for MHC levels by Western blotting. (FIG. 4C) Nilotinib abolishes LLC-induced loss of myotube mass. Myotubes treated as described in FIG. 4B were subjected to immunofluorescence staining of MHC. Diameter of myotubes was measured. (FIG. 4D) Nilotinib does not suppress myoblast differentiation at the dose antagonizing LLC-induced myotube catabolism. Proliferating C2C12 myoblasts were cultured with differentiation medium containing 10 μM SB202190 (SB), 500 nM nilotinib or DMSO for the indicated time periods. MHC content in the cell lysates were analyzed by Western blotting at indicated times. * signifies a statistically significant difference (p<0.05) determined by one-way ANOVA.

FIG. 5A-F: Nilotinib ameliorates muscle wasting by abrogating the catabolic response in LLC tumor-bearing mice. Nilotinib or DMSO was administered intraperitoneally (0.5 mg/kg/day) to mice 7 days after LLC cell implantation for 14 days. Mice were euthanized on day 21. The tumor was isolated and weighed, and then the net body weight was measured. Muscle samples were collected immediately for analyses of muscle wasting. (FIG. 5A) Nilotinib abrogates p300 activation and catabolic response in LLC tumor-bearing mice. Catabolic markers in TA muscle lysates were analyzed by Western blotting. (FIG. 5B) Nilotinib prevents body weight loss in LLC-bearing mice. (FIG. 5C) Nilotinib does not affect LLC tumor growth. (FIG. 5D) Nilotinib preserves skeletal muscle function in LLC tumor-bearing mice. Grip strength was measured on the day of euthanasia. (FIG. 5E) Nilotinib attenuates skeletal muscle weight loss in LLC tumor-bearing mice. (FIG. 5F) Nilotinib protects against the loss of myofiber mass in LLC tumor-bearing mice. H&E-stained TA cross sections were analyzed for the myofiber cross-sectional area. * signifies a statistically significant difference (p<0.05) determined by one-way ANOVA (FIGS. 5A-E) or Chi-square test (FIG. 5F).

FIG. 6A-B: Nilotinib prolongs survival of mice bearing pancreatic cancer by impeding the development of muscle wasting. Five days after orthotopic implant of KPC cells to mice, nilotinib (0.5 mg/kg/day) or DMSO were administered intraperitoneally until all tumor-bearing mice reached predetermined end point. (FIG. 6A) Nilotinib prolongs survival of mice bearing KPC tumor. Survival of KPC tumor-bearing mice was recorded and analyzed using the Kaplan-Meier survival curve. (FIG. 6B) Nilotinib impedes the loss of muscle strength in mice bearing KPC tumor. Forelimb grip strength of the mice was monitored over the course of the survival study. Data were analyzed by 2-way ANOVA. * signifies a statistically significant difference (p<0.05).

FIG. 7: A graphic illustration of the central role of p38β MAKP in mediating muscle catabolism in response to TLR4 activation based on data from the current and previous studies.

FIG. 8: Nilotinib inhibits LLC cell condition medium-induced UBR2 upregulation in myotubes. C2C12 myotubes were pretreated with nilotinib at indicated concentrations or vehicle (0.2% DMSO) for 30 min, and then treated with LCM for 8 hours. UBR2 levels were analyzed by Western blotting, quantified by optical density (OD).

FIG. 9: Nilotinib inhibits p38 MAPK activation and downstream catabolic response in primary human myotubes treated with conditioned medium of H1299 cells. Primary human myotubes were pretreated with nilotinib (500 nM) or vehicle for 30 min followed by treatment with human lung carcinoma H1299 cell-conditioned medium, or conditioned medium of nontumorigenic NL20 cells as control. Activation of p38 MAPK (1 h), phosphorylation of C/EBPb on Thr-188 (1 h), and UBR2 levels (8 h) were analyzed by Western blotting.

FIG. 10: Nilotinib does not alter the growth of KPC tumor. At the end point, both of DMSO or nilotinib-treated KPC tumor-bearing mice were euthanized. Tumors were isolated and weighted (left panel). Net body weight was measured (right panel).

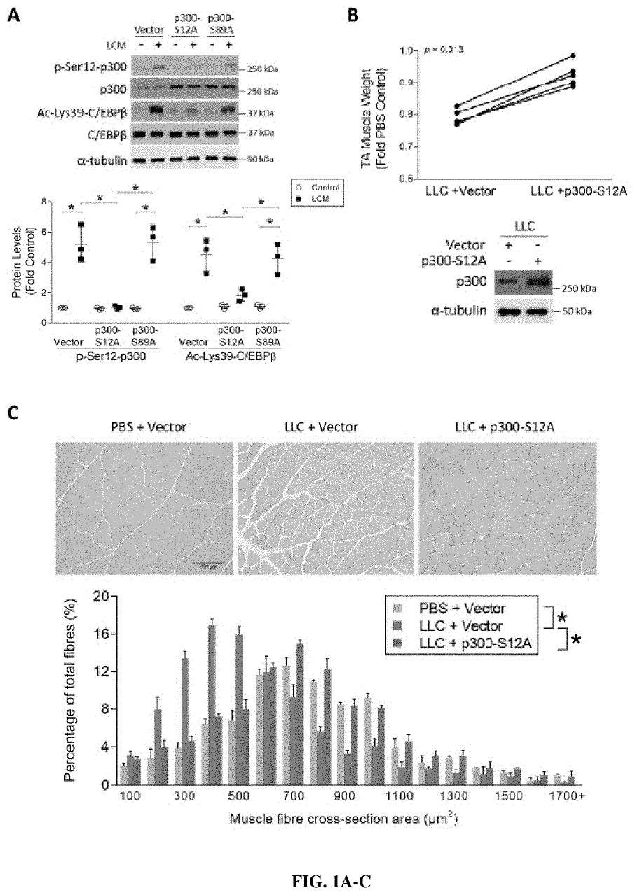

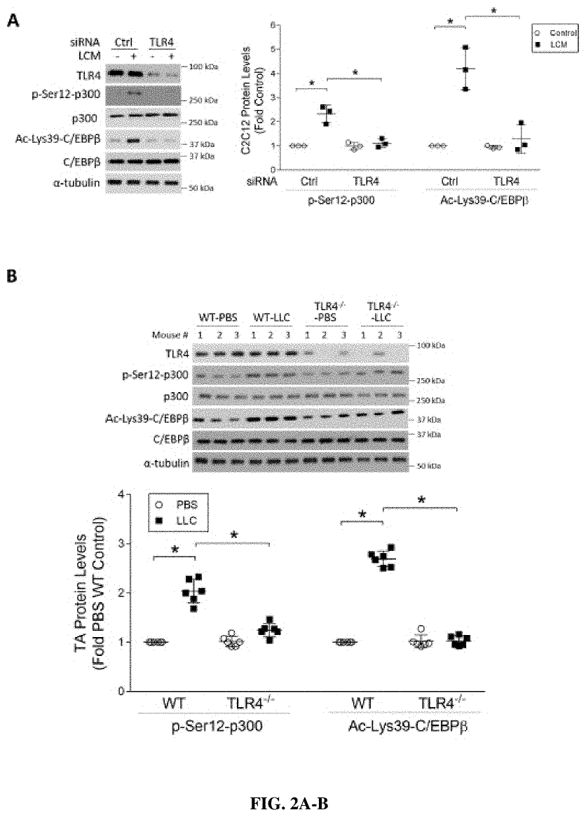

### DESCRIPTION OF ILLUSTRATIVE EMBODIMENTS

### I. Definitions

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

As used herein, and unless otherwise indicated, the term disease or disorder includes muscle wasting diseases and disorders, including, but, not limited to: the muscle wasting associated with cancer; chronic heart failure; chronic kidney disease; Chronic Obstructive Pulmonary Disease; neuromuscular diseases (such as, but, not limited to Amyotrophic Lateral Sclerosis; Muscular Dystrophy; Multiple Sclerosis; spinal muscular atrophy) prolonged inactivity; malnutrition and sarcopenia.

As used herein, and unless otherwise indicated, the terms “treat,” “treating,” “treatment” and “therapy” contemplate an action that occurs while a patient is suffering from a disorder that involves muscle wasting and which reduces the severity of one or more symptoms or effect of such a disorder.

Where the context allows, the terms “treat,” “treating,” and “treatment” also refers to actions taken toward ensuring that individuals at increased risk of a disorder that involves muscle wasting and which reduces the severity are able to receive appropriate surgical and/or other medical intervention prior to onset of muscle wasting and which reduces the severity.

As used herein, and unless otherwise indicated, the terms “prevent,” “preventing,” and “prevention” contemplate an action that occurs before a patient begins to suffer from a disorder that involves that involves muscle wasting, that delays the onset of, and/or inhibits or reduces the severity of a disorder that involves muscle wasting.

As used herein, and unless otherwise indicated, the terms “manage,” “managing,” and “management” encompass preventing, delaying, or reducing the severity of a recurrence of a disorder that involves muscle wasting in a patient who has already suffered from such a disease, disorder or condition. The terms encompass modulating the threshold, development, and/or duration of the disorder that involves muscle wasting.

As used herein, and unless otherwise specified, a “therapeutically effective amount” of a compound is an amount sufficient to provide any therapeutic benefit in the treatment or management of a disorder that involves muscle wasting or to delay or minimize one or more symptoms associated with a disorder that involves muscle wasting. A therapeutically effective amount of a compound means an amount of the compound, alone or in combination with one or more other therapies and/or therapeutic agents that provide any therapeutic benefit in the treatment or management of a disorder that involves muscle wasting. The term “therapeutically effective amount” can encompass an amount that alleviates a disorder that muscle wasting, improves or reduces a disorder that involves muscle wasting or improves overall therapy, or enhances the therapeutic efficacy of another therapeutic agent.

As used herein, and unless otherwise specified, a “prophylactically effective amount” of a compound is an amount sufficient to prevent or delay the onset of a disorder that involves muscle wasting or one or more symptoms associated with a disorder that involves muscle wasting. A prophylactically effective amount of a compound means an amount of the compound, alone or in combination with one or more other treatment and/or prophylactic agent that provides a prophylactic benefit in the prevention of a disorder that involves muscle wasting. The term “prophylactically effective amount” can encompass an amount that prevents a disorder that involves muscle wasting, improves overall prophylaxis, or enhances the prophylactic efficacy of another prophylactic agent. The “prophylactically effective amount” can be prescribed prior to, for example, the development of a disorder that involves muscle wasting.

As used herein, “patient” or “subject” includes mammalian organisms which are capable of suffering from a disorder that involves muscle wasting, as described herein, such as human and non-human mammals, for example, but not limited to, rodents, mice, rats, non-human primates, companion animals such as dogs and cats as well as livestock, e.g., sheep, cow, horse, etc.

As used herein, “patient” or “subject” includes mammalian organisms which are capable of developing muscle wasting conditions, such as, but not limited to as cachexia, chronic heart failure; chronic kidney disease; Chronic Obstructive Pulmonary Disease; neuromuscular diseases (such as, but, not limited to Amyotrophic Lateral Sclerosis; Muscular Dystrophy; Multiple Sclerosis; spinal muscular atrophy) prolonged inactivity; malnutrition and sarcopenia.

### II. The Present Embodiments

Cancer stimulates muscle wasting through complex signaling mechanisms, which can be targeted for therapeutic purpose. The inventor has previously determined that cachexia-inducing cancers release high levels of Hsp70 and Hsp90 through extracellular vesicles, which activate TLR4 on skeletal muscle cells to induce muscle catabolism directly, and activate TLR4 systemically to increase circulating inflammatory cytokines such as TNFα and IL-6 that also promote muscle catabolism indirectly. Activation of TLR4 and receptors for inflammatory cytokines such as TNFα and IL-6 promote muscle protein loss through the activation of the 0 isoform of p38 MAPK as described in the Examples below, it is shown that cancer induces p300 activation through TLR4-mediated activation of p38β MAPK and p300. Particularly, p38β MAPK mediates cancer-induced muscle catabolism through activation of p300 and thus inhibiting p38β MAPK provides an effective therapeutic strategy for intervening cancer-induced muscle wasting. More recently, the inventor collected muscle samples from cancer patients and found that p38β MAPK activation correlates to muscle wasting, supporting it as a therapeutic target of cancer cachexia. As demonstrated herein the protein kinase inhibitor nilotinib (Tasigna®, Novartis Pharmaceuticals), an FDA-approved therapeutic agent can be used to inhibit p38β MAPK activity and thus muscle protein loss, such as but not limited to that which is associated with cancer cachexia. This may be due to the significantly higher binding affinity for p38β MAPK than for p38α MAPK or even the original target BCR-Abl for which it was developed as a therapeutic agent for chronic myelogenous leukemia ¹³.

It was determined that nilotinib inhibits p38β MAPK mediated activation of p300 and C/EBPβ in skeletal muscle cells at a concentration approximately 20-fold lower than the p38α/α MAPK dual inhibitor SB202190. Thus, the use of nilotinib would not be anticipated to inhibit myogenic differentiation, which is mediated by p38α MAPK and should be avoided. As exemplified below, systemic administration of nilotinib to diverse types of tumor-bearing mice at a dose that is at least 30 times lower than that for treatment of mouse leukemia models alleviated muscle wasting and prolonged lifespan. On the other hand, it is demonstrated below that the effective dose of nilotinib did not inhibit myogenic differentiation as SB202190 did. Therefore, cancer-induced muscle wasting can be effectively treated by inhibiting p38β MAPK through repurposing nilotinib.

Evidence is provided here that p38β MAPK is an indispensable upstream signaling molecule for the activation of the acetyltransferase activity of p300 through site-specific phosphorylation in response to TLR4 activation that causes muscle wasting. These data indicate that p38β MAPK orchestrates multiple intricate signaling events that are required for the activation of the muscle catabolism machinery in response to a cancer burden. This study not only reiterates the key role of p38β MAPK in mediating muscle wasting, and in particular cancer-induced muscle wasting, but reconciles the previous observations that both p300 and p38β MAPK are essential for the development of muscle wasting. It is also demonstrated that selective inhibition of p38β MAPK with an FDA-approved drug for cancer treatment, nilotinib, alleviates muscle wasting in tumor-bearing mice. Thus, the current study has made significant conceptual advances in the underlying etiology of muscle wasting and in particular cancer-induced cachexia.

There is a lack of an established treatment for cancer cachexia, thus there is a longstanding and unmet medical need for a therapeutic directed at this lethal disorder. Provided herein, is data demonstrating that nilotinib is an intervention applicable to cancer-induced cachexia. As with all kinase inhibitors, relative selectivity is dependent on concentration. Nilotinib's high binding affinity to p38β MAPK (Kd 32 nM) ¹³, it is demonstrated that it completely abrogates p38β MAPK-mediated p300 activation even at a very low concentration (500 nM) and is approximately 20-fold more potent than the p38α/α MAPK dual inhibitor SB202190, resulting in preservation of myofibrillar protein myosin heavy chain that is highly susceptible to cancer cachexia. Importantly, at this concentration nilotinib does not inhibit p38α MAPK-mediated myogenic differentiation, thus rendering it selective for p38β MAPK. Nilotinib, is an FDA-approved drug for chronic myelogenous leukemia which is believed to act by inhibiting the BCR-Abl kinase (Kd 56-62 nM). In mouse models of leukemia using therapeutic doses ranging from 15 to 75 mg/kg/day off-target binding occurs with a number of kinases and thus ¹³, therapeutic doses of nilotinib for leukemia inevitably cause a number of adverse effects (hcp.novartis.com/tasigna/safety). In contrast, the dose of nilotinib used to effectively alleviate muscle wasting in mouse models of cancer cachexia (0.5 mg/kg/day) is at minimum 30-fold lower than the therapeutic doses for leukemias; hence, selective inhibition of p38β MAPK with a reduction of adverse effects can be achieved, indicating that low dose nilotinib can be a safe intervention for cancer cachexia. In light of the involvement of p38β MAPK in inflammatory signaling 4, nilotinib can be effective for remedying muscle wasting associated with many pathological conditions. For example, in mice bacterial endotoxin-induced muscle wasting (a symptom of sepsis) is alleviated by the inhibition of p38 MAPK.

### III. p38β MAPK and Inhibitors Thereof

p38β mitogen-activated protein kinase, also known as MAPK11 is an enzyme that in humans is encoded by the MAPK11 gene. MAP kinases act as an integration point for multiple biochemical signals and are involved in a wide variety of cellular processes such as proliferation, differentiation, transcription regulation, and development. This kinase is most closely related to p38 MAP kinase, both of which can be activated by proinflammatory cytokines and environmental stress. This kinase is activated through its phosphorylation by MAP kinase kinases (MKKs), preferably by MKK6. Transcription factor ATF2/CREB2 has been shown to be a substrate of this kinase. MAPK11 has been shown to interact with HDAC3 and Promyelocytic leukemia protein.

Inhibitors of p38β MAPK, either specific or pan-MAPK inhibitors, include Nilotinib, Pamapimod, BIRB 796 (Doramapimod), SB202190, SB203580, SD 169, R1487, SB242235 and D4476.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Cell cultures. Murine C2C12 myoblasts (American Type Culture Collection, ATCC) and human skeletal myoblasts (GIBCO®) were grown in growth medium (DMEM supplemented with 10% fetal bovine serum) at 37° C. under 5% CO₂. Myoblast differentiation was induced at 85% confluence with differentiation medium (DMEM supplemented with 4% heat-inactivated horse serum in) for 96 hrs. Preconditioned medium from 48-hour cultures of Lewis lung carcinoma cells (National Cancer Institute, Frederick, Md.), H1299 human lung carcinoma cells (ATCC) or KPC cells (a gift from Dr. Elizabeth Jaffee of Johns Hopkins University) 14 were collected and centrifuged (1000×g, 5 min). Conditioned medium of non-tumorigenic NL20 cells (human lung epithelial cells, ATCC) was used as control. The supernatant was used to treat myotubes (25% final volume in fresh medium) when indicated and replaced every 24 h. When indicated, myotubes were pre-treated with nilotinib (CDS023093, Sigma, St. Louis, Mo.) for 30 mins at doses ranging from 10 nM to 10 μM. All cell lines were tested negative for mycoplasma contamination. Cell culture-based experiments were replicated independently for 3 times.

Transfection of siRNA and plasmids in C2C12 myotubes. At 60% confluence, C2C12 myoblasts were transfected with siRNA targeting p38α (SASI_Mm01_00020743, Sigma) or p38β MAPK (SASI_Mm01_00044863, Sigma) or scramble control siRNA (Ambion, Austin, Tex.). For overexpression studies, myoblasts were transfected with plasmids encoding phosphorylation-defective p300 mutants ¹⁵ or constitutive active mutants of p38α or p38β MAPK ¹⁶. All transfections were performed with jetPRIME reagent (Polyplus-transfection Inc., Illkirch, France) according to the manufacturer's protocol. Growth medium was replaced with differentiating medium 24 hours after transfection and differentiation was induced as described earlier.

Animal use. Experimental protocols were pre-approved by the institutional Animal Welfare Committee at the University of Texas Health Science Center at Houston. LLC cells (1×10⁶ in 100 W) were injected subcutaneously into the flanks of 8-week-old male C57B16 mice, TLR4^{-/-} mice in the C57BL/6 background ¹⁷, or p38β MAPK muscle-specific knockout and p38β MAPK-floxed mice in the C57BL/6 background ¹⁸. Nilotinib treatment was initiated seven days after LLC cell implantation when palpable tumor was detected and administered via the intraperitoneal route (0.5 mg/kg/day prepared in 50% DMSO in PBS). DMSO was injected accordingly as vehicle control. Plasmid encoding the p300-S12A mutant was transfected into TA muscle by electroporation on day 7 and day 14 following LLC implantation as described previously ¹². The contralateral TA was transfected with an empty vector as control. Development of cachexia was monitored by body weight and forelimb grip strength test, and usually took place within 21 days after implantation. On day 21, mice were euthanized, and muscle samples were harvested for further analyses. For survival study, orthotopic implantation of KPC cells was performed based on the procedures by Zhu et al. ¹⁹. Briefly, a longitudinal incision was made to open the abdominal cavity for pancreas exposure. Then, 2×10⁶ KPC cells suspended in 20 μl PBS were injected to the tail of pancreas. Nilotinib (0.5 mg/kg/day) was administered daily 5 days after tumor implant until predetermined end point was reached.

Western blotting. All procedures were adhered to our previous publication ¹². The following primary antibodies were used: anti-p300 (1:500, sc584, Santa Cruz), anti-C/EBPβ (1:1000, MA1-827, Thermo Fisher), anti-pT188-C/EBPβ (1:1000, 3084, Cell Signaling Technology), anti-TLR4 (1:500, sc16240, Santa Cruz), anti-p38α MAPK (1:500, sc271120, Santa Cruz), anti-p38s MAPK (1:500, 2339, Cell Signaling Technology), anti-p38 MAPK (1:1000, 9212, Cell Signaling Technology), anti-p-p38 MAPK (1:1000, 4511, Cell Signaling Technology), anti-MAFbx (1:1000, AP2041, ECM Bioscience), anti-UBR2 (1:500, NBP1-45243, Novus Biologicals), anti-LC3 (1:2000, NB100-2220, Novus Biologicals) and anti-MHC (1:1000, MAB4470, R&D Systems). Antibody against acetylated Lys-39 of C/EBPβ (1:2000) was generated as previously described ¹². Antibody against phosphorylated Ser-12 of p300 (1:1500) were generated by Pocono Rabbit Farm & Laboratory (Canadensis, Pa.) from rabbit using the peptide PGPPS(P)AKRPKLSSPAC (SEQ ID NO: 1). Data were normalized to α-Tubulin (Development Studies Hybridoma Bank at the University of Iowa, Iowa City, Iowa).

Fluorescence microscopy and histology study. C2C12 myotubes were stained with anti-MHC antibody (1:1000, MAB4470, R&D Systems) and anti-mouse Alexa Fluor® Plus 488 secondary antibody (1:200, A32723, ThermoFisher), and examined using a Zeiss Axioskop 40 microscope and a Zeiss Axiocam MRM camera system controlled by Axiovision Release 4.6 imaging software. Acquired images were edited using the Photoshop software. Myotube diameter was measured in MHC-stained myotubes as previously described ¹². Cross-sectional area of H&E-stained muscle sections was quantified by using the ImageJ software (NIH). Approximately 100 myofibers from each of 5 random views were quantified.

Immunoprecipitation. Immunoprecipitation of p38 MAPK from myotube lysate (1 mg of protein) was performed using an anti-p38 MAPK antibody (1:100; CS9212, Cell Signaling) as described previously ⁸.

Statistical analyses. Statistical analyses were conducted using the SPSS 22.0 software package (IBM, Chicago, Ill.). Data distributions were confirmed by the normality test. All data were expressed as means±standard deviation (SD). Comparisons were made by one-way ANOVA followed by Tukey post-hoc test, Paired t-test, Chi-Square test and two-way ANOVA as appropriate. Statistical significance was accepted at p<0.05.

### Example 2—Results

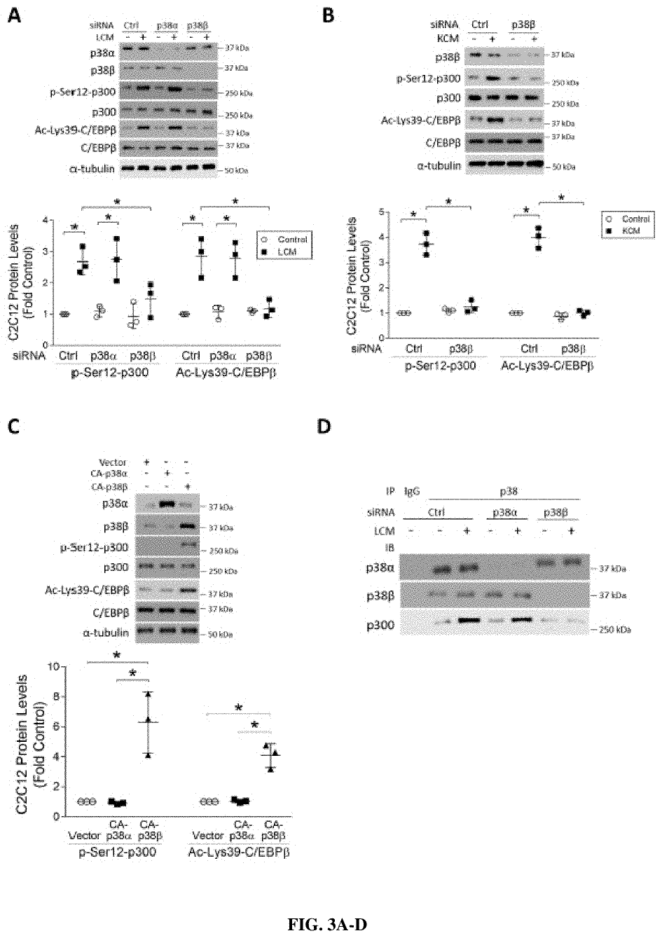

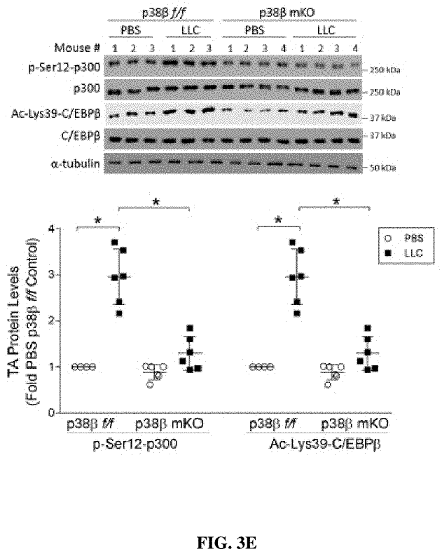

p38β MAPK mediates p300 activation by diverse types of cancer cells. To identify the TLR4 effector that mediates Ser-12 phosphorylation of p300, the inventor characterized the role of p38β MAPK based on that as a protein kinase downstream of TLR4 it is essential for tumor-induced muscle catabolism. Utilizing siRNA-mediated knockdown of p38α or p38β MAPK it was observed that only p38β MAPK, but not p38α MAPK, was critical to LCM-induced p300 phosphorylation on Ser 12 and C/EBPβ acetylation on Lys 39 in myotubes (FIG. 3A). Given that p38β MAPK-mediated muscle catabolism is also activated in human pancreatic ductal adenocarcinoma (PDAC) cell lines (AsPC-1 and BxPC-3) that are highly cachectic, it was identified that p38β MAPK also activates p300 in response to pancreatic cancer, by utilizing the mouse PDAC cell line KPC derived from the original KPC mouse line with knock-in pancreas specific alleles KRAS^{G12D} and P53^{R172H} via the Pdx1-Cre^{+/+} driver 3 by backcrossing the mice to the C57BL/6 background ¹⁴. Treatment of myotubes with conditioned medium of KPC cells (KCM) resulted in increased Ser-12 phosphorylation of p300 as well as Lys-39 acetylation of C/EBPβ, both of which were abrogated in p38β MAPK-deficient myotubes (FIG. 3B), indicating that diverse types of cachexia-inducing cancer cells activate the acetyltransferase activity of p300 in a p38β MAPK-dependent manner. Conversely, over-expression of a constitutively active mutant of p38β MAPK ¹⁶ in myotubes, but not that of p38α MAPK, recapitulated the site-specific p300 phosphorylation and C/EBPβ acetylation seen in LCM/KCM-treated myotubes (FIG. 3C). Using immunoprecipitation to pull down total p38 MAPK from myotube lysate, it was determined that p300 was co-precipitated with p38 MAPK at baseline, which was increased dramatically in response to LCM. By contrast, this elevation was abrogated in myotubes that are deficient in p38p MAPK, but not p38α MAPK, indicating that LCM-activated p38β MAPK specifically interacts with p300, resulting in its phosphorylation on Ser-12 (FIG. 3D). To verify that p38β MAPK mediates the phosphorylation and activation of p300 in vivo, it was determined that in p38β MAPK muscle-specific knockout mice (p38β mKO) that are resistant to LLC-induced muscle wasting ¹¹ the LLC tumor failed to induce p300 phosphorylation on Ser-12 and C/EBPβ acetylation on Lys-39 in TA muscle (FIG. 3E). These data indicate that p38β MAPK is a key mediator of cancer-induced muscle catabolism due to its activation of the p300-C/EBPβ signaling pathway in response to TLR4 activation.

Nilotinib protects against LLC tumor-induced muscle wasting by selective inhibition of p38β MAPK. The p38 MAPK family has four members with distinctive functions, of which three are expressed in skeletal muscle (α, β and γ) ²⁴. Existing p38 MAPK inhibitors are either p38α/β dual inhibitors or p38α-specific inhibitors, which are not suitable for intervening in cancer cachexia and other muscle wasting disorders, due to the essential role of p38α MAPK in myogenic differentiation ^{25, 26}. Inhibiting p38α MAPK would impede the regeneration of cachectic muscle. In addition, p38α MAPK, but not p38β MAPK, is responsible for many of the known biological activities of p38 MAPK ^{29, 30}. Thus, it is necessary to have a protein kinase inhibitor that is selective for p38β MAPK and is approved for human consumption in the intervention of cancer cachexia. The small molecule BCR-Abl kinase inhibitor nilotinib is an FDA-approved therapy for chronic myelogenous leukemia and exhibits 3-fold higher binding affinity for p38β MAPK than p38α MAPK ¹³, making it the only relatively p38β MAPK-selective inhibitor available for human use. Importantly, the binding affinity of nilotinib for p38β MAPK (Kd 32 nM) is about twice of that for its originally intended target BCR-Abl (Kd 56-62 nM) ¹³, indicating that lower doses of nilotinib are sufficient to inhibit p38β MAPK effectively, and hence, fewer adverse effects and lower toxicity is expected.

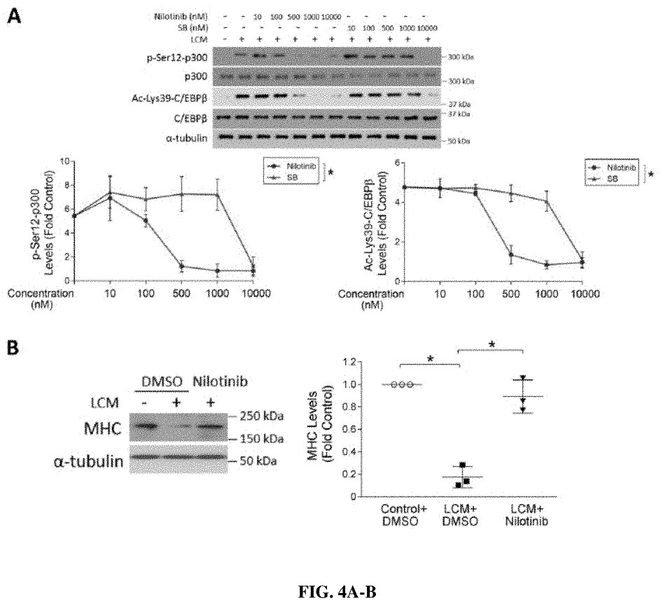

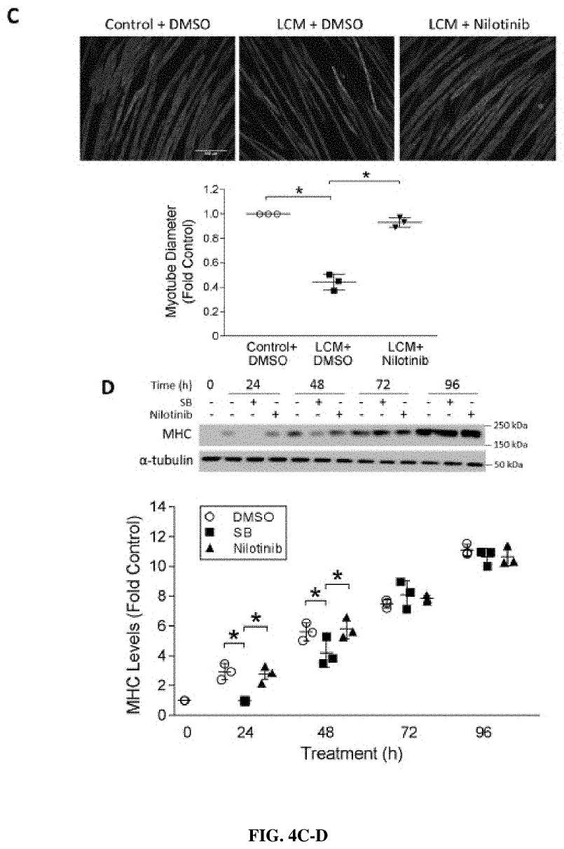

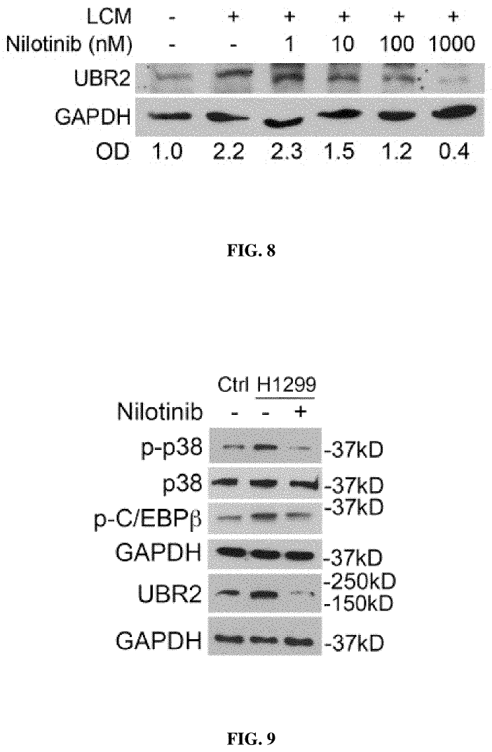

A concentration-activity study in C2C12 myotubes revealed that nilotinib inhibited LCM-induced p300 phosphorylation on Ser-12 in a concentration-dependent manner. Notably, nilotinib totally abolished this reaction at approximately 500 nM, which was about 20-fold more potent than SB202190, a p38α/β dual inhibitor that attenuates LLC tumor-induced muscle wasting in mice 8. Concordantly, C/EBPβ acetylation on Lys-39 was inhibited by nilotinib in a similar manner (FIG. 4A). These results indicate that nilotinib is highly efficacious in inhibiting the activation of the acetyltransferase activity of p300 by LLC. Consequently, nilotinib inhibited LCM-induced upregulation of C/EBPβ-controlled E3 ligase UBR2 ¹⁰ in C2C12 myotubes in a similar concentration-dependent manner, confirming the inhibition of C/EBPβ-mediated catabolic signaling by nilotinib (FIG. 8). To assess whether nilotinib inhibits myotube p38 MAPK activation by cancer in general, the inventor observed that pretreating primary human myotubes with 500 nM of nilotinib abrogated p38 MAPK activation by conditioned medium of the human lung carcinoma cell line H1299, resulting in a blockade of p38β MAPK-mediated C/EBPβ phosphorylation on Thr-188 ⁹ and upregulation of E3 ligase UBR2 ¹⁰ (FIG. 9). This result suggests that nilotinib inhibits a human cancer-induced p38 MAPK activation and the ensuing catabolic response in human muscle cells. Consequently, C2C12 myotubes treated with 500 nM nilotinib were resistant to LCM-induced loss of myofibrillar protein myosin heavy chain (MHC) (FIG. 4B) and myotube mass as measured by myotube diameters (FIG. 4C). These effects are comparable to the effects of 10 μM of SB202190 as reported previously 8, demonstrating the high efficacy of nilotinib in inhibiting p38β MAPK-mediated muscle catabolism.

To demonstrate the specificity of nilotinib inhibition of p38β MAPK, the effect of nilotinib on myogenic differentiation which requires p38α MAPK was determined. C2C12 myoblasts were allowed to differentiate for 96 hours in the presence of 500 nM of nilotinib or 10 μM of SB202190. As anticipated, the duel inhibitor SB202190 delayed the p38α MAPK dependent onset of myoblast differentiation as indicated by significantly lower expression of MHC at 24 and 48 hours in comparison to control cells. In contrast, nilotinib did not alter MHC expression over the course of differentiation (FIG. 4D). This indicated that at 500 nM nilotinib selectively inhibits p38β MAPK without affecting p38α MAPK. 500 nM of nilotinib exerted maximum inhibition of p300 activation as shown in FIG. 4A, which means that the therapeutic dose of nilotinib for cancer-induced muscle wasting would be comfortably within its p38β MAPK-selective dose range.

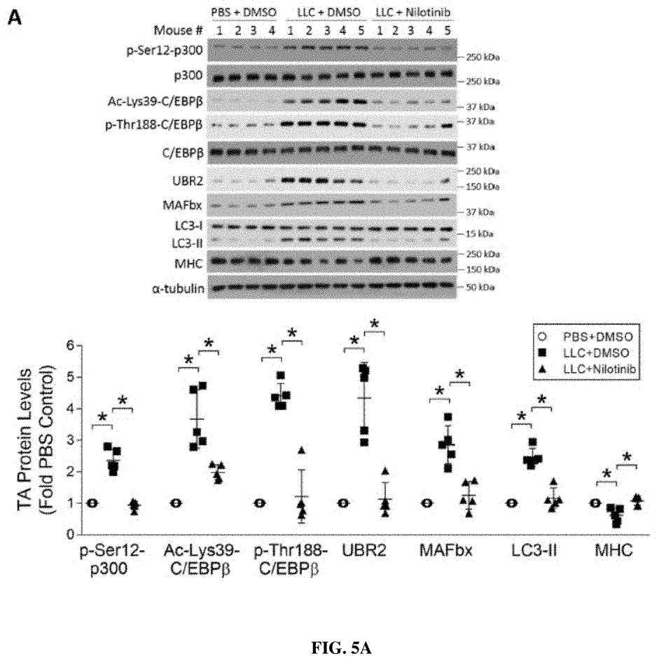

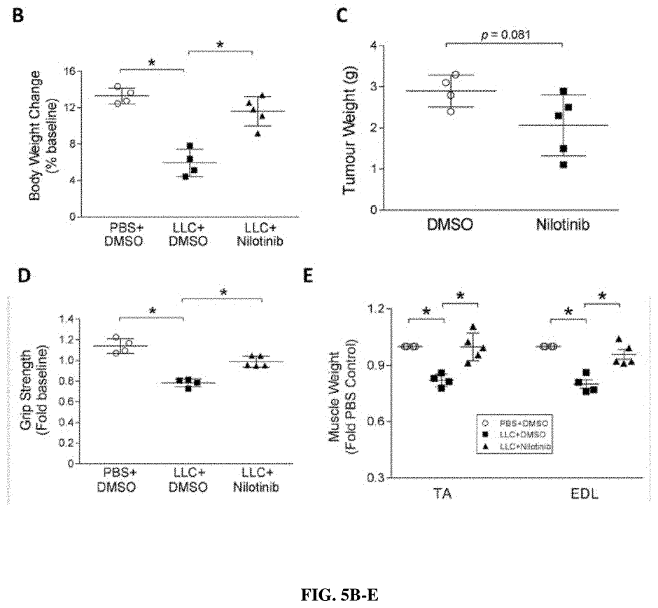

To demonstrate that nilotinib ameliorates cancer-induced muscle wasting in vivo, nilotinib was administered intraperitoneally to LLC tumor-bearing mice at a dose of 0.5 mg/kg/day from day 7 for 2 weeks. Similar to its effects in vitro, nilotinib abolished the activation of p300 measured as its Ser-12 phosphorylation and subsequent Lys-39 acetylation of C/EBPβ, resulting in a blockade of the upregulation of C/EBPβ-controlled E3 ligases UBR2 and atroginl/MAFbx, the increase in autophagosome formation as measured by LC3-II levels, and the loss of MHC in LLC tumor-bearing mice (FIG. 5A). Consequently, nilotinib protected against the loss of body weight in LLC-bearing mice (FIG. 5B) without affecting tumor growth (FIG. 5C). Nilotinib also attenuated LLC-induced loss of muscle strength (FIG. 5D) as well as loss of TA and EDL weight (FIG. 5E). Finally, measurement of myofiber cross-sectional area in TA confirmed that nilotinib preserved myofiber mass in LLC tumor-bearing mice (FIG. 5F). These data indicate that nilotinib protects against cancer-induced muscle wasting.

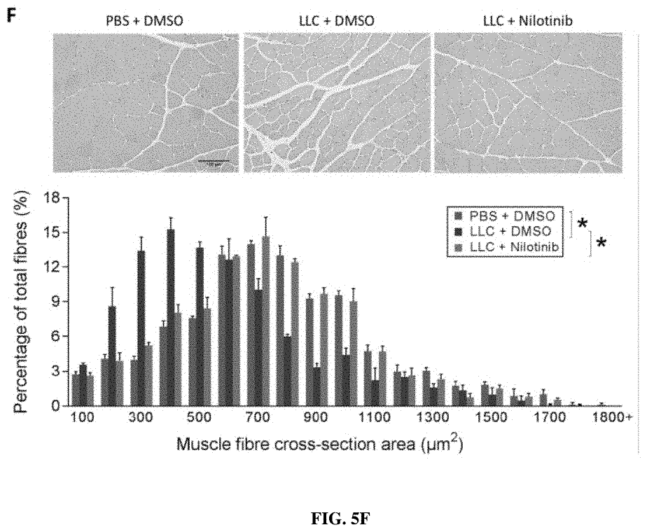

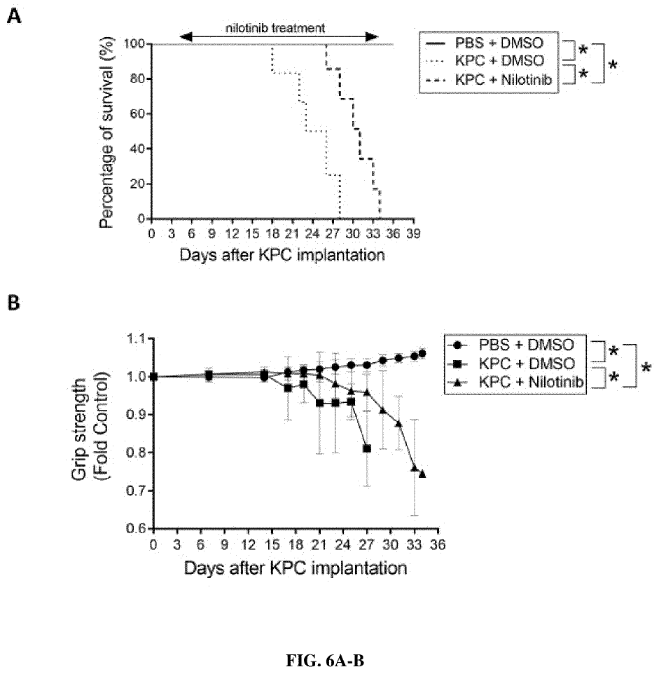

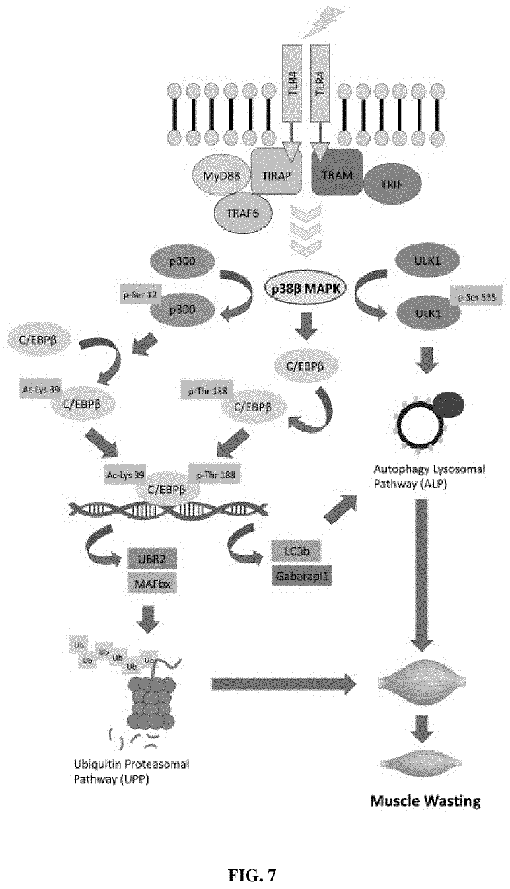

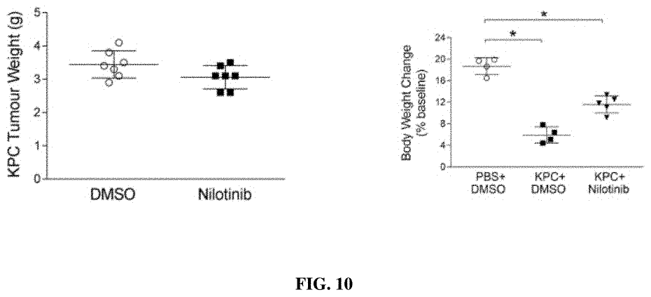

Nilotinib prolongs survival of KPC tumor-bearing mice. The ultimate goal of managing cancer cachexia is to prolong patient survival. To demonstrate that nilotinib treatment prolongs the survival by limiting cancer cachexia, a study was done using a syngeneic pancreatic cancer model by orthotopically implanting the KPC cells ¹⁴ to C57BL/6 mice ¹⁹ for two reasons. First, the LLC model is generated by subcutaneous implantation of LLC cells and frequently develops skin ulceration, making it infeasible for prolonged study that comply with animal welfare concerns. Second, patients with pancreatic cancer have the highest prevalence and severity of cachexia among all cancer patients ⁴ with a 5-year survival rate under 10%; and nearly 80% of deaths in patients with advanced pancreatic cancer are associated with severe wasting ³¹⁻³⁴. The ability of nilotinib prolongs survival of mice bearing pancreatic cancer, it would provide strong evidence that it should extend survival in human's inflicted with cancer cachexia. As shown in FIG. 6A, mice bearing KPC tumor treated with DMSO (vehicle for nilotinib) died between 18 to 28 days after implant (median survival 24.5 days). However, KPC tumor-bearing mice treated with nilotinib survived significantly longer, i.e., 26 to 34 days (median survival 31 days). Over this course, nilotinib treatment attenuated the loss of muscle function measured as grip strength (FIG. 6B), suggesting that the extension of survival by nilotinib treatment is due to the alleviation of muscle wasting. At the end point, however, both DMSO and nilotinib-treated groups developed advanced muscle wasting without significant difference in tumor volume (FIG. 10), suggesting that nilotinib prolonged survival of KPC tumor-bearing mice by impeding the initiation and progression of muscle wasting. These data demonstrate that the morbidity and mortality of mice bearing pancreatic cancer can be ameliorated by selective inhibition of p38β MAPK and supports the use of nilotinib as an anti-cachexia therapeutic.

Systemic administration of nilotinib to diverse types of tumor-bearing mice at a dose that is at least 30 times lower than that for treatment of mouse models of leukemia alleviated muscle wasting and prolonged lifespan, indicating that cancer-induced muscle wasting could be effectively treated by inhibiting p38β MAPK through repurposing nilotinib.

### Example 3—Prophetic Example

The use of inhibitors of p38β MAPK activity to treat humans. Clinical trials for the use of p38β MAPK inhibitors such as nilotinib for treatment muscle wasting diseases or disorders, would utilize a much lower dose than for its previous approval for leukemia.

For example, the serum concentration of nilotinib 24 hours after first PO administration in humans of 50 mg resulted in a serum concentration of approximately 400 ng/ml. As described herein 500 nM abrogated muscle protein loss, which it the equivalent of 265 ng/ml, therefore an effective therapeutic human dose may be in the range of 50 mg/day, and it is anticipated that after 5 doses the blood concentration will rise to an effective higher and stable level. Such trials would include dosages ranging from 10 mg-200 mg of nilotinib taken orally, twice a day (PO BID) with a preferred range encompassing 50 mg, 55 mg, 60 mg, 65 mg, 70 mg, 75 mg, 80 mg, 85 mg, 90, mg, 95 mg, 100 mg, 105 mg 110 mg, 115 mg, 120 mg, 125 mg, 130 mg 135 mg, 140 mg, 145 mg, 150 mg, 155 mg, 160 mg, 165 mg, 170 mg, 175 mg, 180 mg, 185 mg, 190, mg, 195 mg, and 200 mg of nilotinib PO BID (which is still lower than the standard dose for CML 300 mg PO BID). Therefore, in an included embodiment is the method of using nilotinib for treatment muscle wasting diseases or disorders in humans.

example, the serum concentration of nilotinib 24 hours after first PO administration in humans of 50 mg resulted in a serum concentration of approximately 400 ng/ml. As described herein 500 nM abrogated muscle protein loss, which was 265 ng/ml, therefore an effective therapeutic human dose would be in the range of 50 mg/day, and it is anticipated that after 5 doses the blood concentration will rise to an effective higher and stable level. Such trials may include dosages that fall within the range of 100 mg/day (which is still 6 times lower than the standard dose for CML 300 mg PO BID), 75 mg/day, 50 mg/day, 25 mg/day and 10 mg/day.

Such trials might involve pancreatic cancer patients as over 80% of them develop cachexia. Another group of patients might be those with gastrointestinal cancers (60% develop cachexia) and/or lung cancer patients (50% develop cachexia), future trials might be expanded to include muscle wasting associated with diseases such as COPD, CHF, subsequent to surgery or burns and muscle wasting associated with aging.

In view of the foregoing findings which demonstrate that the inhibition of p38β MAPK function by low dose nilotinib results in, inter alia, a decrease in muscle wasting as associated with cachexia. While the in vivo data presented in the above examples was obtained using mice, those skilled in the art will readily recognize that these observations extend to other mammals including humans when the appropriate inhibitors are utilized. In fact, the inventor has collected muscle samples from cancer patients and found a similar activation of p38β MAPK and downstream events in cachectic muscle. The findings described in this application further indicate that inhibitors (inter alia, antibodies, proteins, polypeptides, peptides or fragments thereof, genetic disruption by recombination, RNAi, aptamers, small molecule inhibitors or any other form of inhibitor known to the art) of p38β MAPK can reproduce, the physiologic effects observed in mice in which p38β MAPK activity has been disrupted using nilotinib. This disclosure clearly demonstrates that nilotinib, a selective inhibitor of p38β MAPK, can be used to treat p38β MAPK mediated muscle wasting diseases and disorders, such as but not limited to cancer cachexia; chronic heart failure; chronic kidney disease; Chronic Obstructive Pulmonary Disease; neuromuscular diseases (such as, but, not limited to Amyotrophic Lateral Sclerosis; Muscular Dystrophy; Multiple Sclerosis; spinal muscular atrophy) prolonged inactivity; malnutrition and muscle loss associated with aging (sarcopenia).

Clinical trials for the use of p38β MAPK inhibitors, such as nilotinib, for treatment muscle wasting diseases or disorders would utilize a much lower dose that for its previous approval for leukemia. Such trials might involve pancreatic cancer patients as over 80% of develop cachexia. Another group of patients might be those with gastrointestinal cancers (60% develop cachexia) and/or lung cancer patients (50% develop cachexia), future trials might be expanded to include muscle wasting associated with diseases, such as, but not limited to, COPD, CHF, subsequent to surgery or burns and sarcopenia.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Fearon K C, Glass D J, Guttridge D C. Cancer cachexia: mediators, signaling, and metabolic pathways. Cell metabolism. 2012; 16: 153-166.
2. Fearon K, Strasser F, Anker S D, et al. Definition and classification of cancer cachexia: an international consensus. Lancet Oncol. 2011; 12: 489-495.
3. Andreyev H J, Norman A R, Oates J, Cunningham D. Why do patients with weight loss have a worse outcome when undergoing chemotherapy for gastrointestinal malignancies? Eur J Cancer. 1998; 34: 503-509.
4. Baracos V E, Martin L, Korc M, Guttridge D C, Fearon K C H. Cancer-associated cachexia. Nat Rev Dis Primers. 2018; 4: 17105.
5. Siegel R L, Miller K D, Jemal A. Cancer statistics, 2019. C A Cancer J Clin. 2019; 69: 7-34.
6. Zhang G, Liu Z, Ding H, Miao H, Garcia J M, Li Y P. Toll-like receptor 4 mediates Lewis lung carcinoma-induced muscle wasting via coordinate activation of protein degradation pathways. Sci Rep. 2017; 7: 2273.
7. Zhang G, Liu Z, Ding H, et al. Tumor induces muscle wasting in mice through releasing extracellular Hsp70 and Hsp90. Nat Commun. 2017; 8: 589.
8. Zhang G, Jin B, Li Y P. C/EBPbeta mediates tumour-induced ubiquitin ligase atroginl/MAFbx upregulation and muscle wasting. The EMBO journal. 2011; 30: 4323-4335.
9. Zhang G, Li Y P. p38beta MAPK upregulates atroginl/MAFbx by specific phosphorylation of C/EBPbeta. Skeletal muscle. 2012; 2: 20.
10. Zhang G, Lin R K, Kwon Y T, Li Y P. Signaling mechanism of tumor cell-induced up-regulation of E3 ubiquitin ligase UBR2. FASEB journal: official publication of the Federation of American Societies for Experimental Biology. 2013; 27: 2893-2901.
11. Liu Z, Sin, K. W. T., Ding, H., Doan, H. A., Gao, S., Miao, H., Wei, Y., Wang, Y., Zhang, G., Li, Y.-P. p38beta MAPK mediates ULK1-dependent induction of autophagy in skeletal muscle of tumor-bearing mice. Cell stress. 2018; 2: 311-324.
12. Sin T K, Zhu J Z, Zhang G, Li Y P. p300 Mediates Muscle Wasting in Lewis Lung Carcinoma.

Cancer Res. 2019; 79: 1331-1342.
13. Manley P W, Drueckes P, Fendrich G, et al. Extended kinase profile and properties of the protein kinase inhibitor nilotinib. Biochimica et biophysica acta. 2010; 1804: 445-453.
14. Foley K, Rucki A A, Xiao Q, et al. Semaphorin 3D autocrine signaling mediates the metastatic role of annexin A2 in pancreatic cancer. Sci Signal. 2015; 8: ra77.
15. Ma H, Guo M, Shan B, Xia Z. Targeted functional analysis of p300 coactivator in Wnt/beta-catenin signaling pathway using phosphoproteomic and biochemical approaches. Journal of proteomics. 2012; 75: 2601-2610.
16. Askari N, Beenstock J, Livnah O, Engelberg D. p38 is Active in vitro and in vivo When Monophosphorylated on Thr180. Biochemistry. 2009; 24.
17. Hoshino K, Takeuchi O, Kawai T, et al. Cutting edge: Toll-like receptor 4 (TLR4)-deficient mice are hyporesponsive to lipopolysaccharide: evidence for TLR4 as the Lps gene product. J Immunol. 1999; 162: 3749-3752.
18. Ding H, Zhang G, Sin K W, et al. Activin A induces skeletal muscle catabolism via p38beta mitogen-activated protein kinase. Journal of cachexia, sarcopenia and muscle. 2017; 8: 202-212.
19. Michaelis K A, Zhu X, Burfeind K G, et al. Establishment and characterization of a novel murine model of pancreatic cancer cachexia. Journal of cachexia, sarcopenia and muscle. 2017.
20. Talbert E E, Cuitino M C, Ladner K J, et al. Modeling Human Cancer-induced Cachexia. Cell Rep. 2019; 28: 1612-1622 e1614.
21. Doyle A, Zhang G, Abdel Fattah E A, Eissa N T, Li Y P. Toll-like receptor 4 mediates lipopolysaccharide-induced muscle catabolism via coordinate activation of ubiquitin-proteasome and autophagy-lysosome pathways. FASEB J. 2011; 25: 99-110.
22. Yang J, Zhang Z, Zhang Y, et al. ZIP4 Promotes Muscle Wasting and Cachexia in Mice with Orthotopic Pancreatic Tumors by Stimulating RAB27B-Regulated Release of Extracellular Vesicles From Cancer Cells. Gastroenterology. 2019; 156: 722-734 e726.
23. Hingorani S R, Wang L, Multani A S, et al. Trp53R172H and KrasG12D cooperate to promote chromosomal instability and widely metastatic pancreatic ductal adenocarcinoma in mice. Cancer Cell. 2005; 7: 469-483.
24. Cuenda A, Rousseau S. p38 MAP-kinases pathway regulation, function and role in human diseases. Biochim Biophys Acta. 2007; 1773: 1358-1375.
25. Perdiguero E, Ruiz-Bonilla V, Gresh L, et al. Genetic analysis of p38 MAP kinases in myogenesis: fundamental role of p38alpha in abrogating myoblast proliferation. EMBO J. 2007; 26: 1245-1256.
26. Palacios D, Mozzetta C, Consalvi S, et al. TNF/p38alpha/polycomb signaling to Pax7 locus in satellite cells links inflammation to the epigenetic control of muscle regeneration. Cell Stem Cell. 2010; 7: 455-469.
27. Ono Y, Sakamoto K. Lipopolysaccharide inhibits myogenic differentiation of C2C12 myoblasts through the Toll-like receptor 4-nuclear factor-kappaB signaling pathway and myoblast-derived tumor necrosis factor-alpha. PLoS One. 2017; 12: e0182040.
28. He W A, Berardi E, Cardillo V M, et al. N F-kappaB-mediated Pax7 dysregulation in the muscle microenvironment promotes cancer cachexia. J Clin Invest. 2013; 123: 4821-4835.
29. Sicard P, Clark J E, Jacquet S, et al. The activation of p38 alpha, and not p38 beta, mitogen-activated protein kinase is required for ischemic preconditioning. Journal of molecular and cellular cardiology. 2010; 48: 1324-1328.
30. O'Keefe S J, Mudgett J S, Cupo S, et al. Chemical genetics define the roles of p38alpha and p38beta in acute and chronic inflammation. J Biol Chem. 2007; 282: 34663-34671.
31. Olive K P, Jacobetz M A, Davidson C J, et al. Inhibition of Hedgehog signaling enhances delivery of chemotherapy in a mouse model of pancreatic cancer. Science. 2009; 324: 1457-1461.
32. Palesty J A, Dudrick S J. What we have learned about cachexia in gastrointestinal cancer.

Digestive diseases. 2003; 21: 198-213.
33. Martignoni M E, Kunze P, Friess H. Cancer cachexia. Molecular cancer. 2003; 2: 36.
34. Wesseltoft-Rao N, Hjermstad M J, Ikdahl T, et al. Comparing two classifications of cancer cachexia and their association with survival in patients with unresected pancreatic cancer.

Nutr Cancer. 2015; 67: 472-480.
35. Lin M G, Hurley J H. Structure and function of the ULK1 complex in autophagy. Current opinion in cell biology. 2016; 39: 61-68.
36. Li Y P, Chen Y, John J, et al. TNF-alpha acts via p38 MAPK to stimulate expression of the ubiquitin ligase atroginl/MAFbx in skeletal muscle. FASEB J. 2005; 19: 362-370.
37. Puppa M J, Gao S, Narsale A A, Carson J A. Skeletal muscle glycoprotein 130's role in Lewis lung carcinoma-induced cachexia. FASEB journal: official publication of the Federation of American Societies for Experimental Biology. 2014; 28: 998-1009.
38. Greco S H, Tomkotter L, Vahle A K, et al. TGF-beta Blockade Reduces Mortality and Metabolic Changes in a Validated Murine Model of Pancreatic Cancer Cachexia. PLoS One. 2015; 10: e0132786.
39. Kumar A, Bhatnagar S, Paul P K. TWEAK and TRAF6 regulate skeletal muscle atrophy. Curr Opin Clin Nutr Metab Care. 2012; 15: 233-239.
40. Erridge C. Endogenous ligands of TLR2 and TLR4: agonists or assistants? Journal of leukocyte biology. 2010; 87: 989-999.
41. Yang H, Wang H, Chavan S S, Andersson U. High Mobility Group Box Protein 1 (HMGB1): The Prototypical Endogenous Danger Molecule. Mol Med. 2015; 21 Suppl 1: S6-S12.
42. Contreras O, Villarreal M, Brandan E. Nilotinib impairs skeletal myogenesis by increasing myoblast proliferation. Skeletal muscle. 2018; 8: 5.
43. Kaur P, Feldhahn N, Zhang B, et al. Nilotinib treatment in mouse models of P190 Bcr/Abl lymphoblastic leukemia. Mol Cancer. 2007; 6: 67.
44. Weisberg E, Catley L, Wright R D, et al. Beneficial effects of combining nilotinib and imatinib in preclinical models of BCR-ABL+ leukemias. Blood. 2007; 109: 2112-2120.

What is claimed is:

1. A method of reducing or preventing muscle loss in a subject comprising administering a composition comprising an effective amount of a p38 MAPK inhibitor to the subject.

2. The method of claim 1, wherein the subject has a muscle wasting disease.

3. The method of claim 2, wherein the subject has a cancer associated with muscle wasting.

4. The method of claim 3, wherein the subject has skeletal muscle wasting, the method further defined as a method for reducing or preventing skeletal muscle loss.

5. The method of claim 3, wherein the subject has cardiac muscle wasting, the method further defined as a method for reducing or preventing cardiac muscle loss.

6. The method of claim 1, wherein the subject has, or has been diagnosed with, cachexia, such as cancer-induced cachexia and/or acute cachexia.

7. The method of claim 1, wherein the subject has, or has been diagnosed with, cachexia induced by kidney failure, heart failure, COPD or arthritis.

8. The method of claim 1, wherein the subject has, or has been diagnosed with, muscular dystrophy, such as Duchenne muscular dystrophy, polymyositis, dermatomyositis, Guillain-Barre syndrome, amyotrophic lateral sclerosis, multiple sclerosis or spinal muscular atrophy.

9. The method of claim 1, wherein the p38 MAPK inhibitor comprises Nilotinib.

10. The method of claim 1, further comprising treating said subject with a second anti-muscle wasting therapy.

11. The method of claim 1, wherein the subject is a human subject or a non-human animal subject.

12. The method of claim 1, wherein said p38 MAPK inhibitor is administered more than once, such as daily, every other day, twice a week, weekly, every other week, or monthly.

13. The method of claim 3, wherein the cancer is breast cancer, ovarian cancer, cervical cancer, testicular cancer, colon cancer, stomach cancer, heady & neck cancer, pancreatic cancer, liver cancer, skin cancer, brain cancer, bladder cancer, bone cancer, lymphoma or leukemia.

14. The method of claim 3, further comprising treating said subject with a second anti-cancer therapy.

15. The method of claim 14, wherein the second anti-cancer therapy is one or more of radiation therapy, chemotherapy, immune therapy, toxin therapy, hormonal therapy and/or surgery.

16. The method of claim 1, wherein the subject is a human subject.

17. A method of treating a subject comprising: (a) identifying a subject having cancer-induced cachexia; and (b) administering a composition comprising an effective amount of a p38 MAPK inhibitor to the subject.

18. The method of claim 17, wherein the p38 MAPK inhibitor comprises Nilotinib.

19. The method of claim 17, wherein the cancer is breast cancer, ovarian cancer, cervical cancer, testicular cancer, colon cancer, stomach cancer, heady & neck cancer, pancreatic cancer, liver cancer, skin cancer, brain cancer, bladder cancer, bone cancer, lymphoma or leukemia.

20. The method of claim 17, further comprising treating said subject with a second anti-cancer therapy.
